(19) United States
(12) Patent Application Publication
(10) Pub. No.: US 2021/0057044 A1
(43) Pub. Date: Feb. 25, 2021

(54) METHOD AND SYSTEM FOR SEQUENCE TYPING USING WHOLE GENOME SEQUENCE DATA WHEN SEQUENCE DATA FOR A GENE MARKER IS MISSING OR UNUSABLE

(71) Applicant: KONINKLIJKE PHILIPS N.V., EINDHOVEN (NL)

(72) Inventors: Reza Sharifi Sedeh, Malden, MA (US); Yu Fan, Cambridge, MA (US); Hareesh Chamarthi, Cambridge, MA (US); Andrew G. Hoss, Cambridge, MA (US)

(21) Appl. No.: 16/924,653

(22) Filed: Jul. 9, 2020

Related U.S. Application Data
(60) Provisional application No. 62/890,656, filed on Aug. 23, 2019.

(51) Int. Cl. G16B 30/00; G16B 40/00

(52) U.S. Cl. CPC G16B 30/00; G16B 40/00

(57) ABSTRACT

A method for sequence typing using whole-genome sequence data, comprising: receiving a plurality of gene marker sets, each gene marker set comprises sequence data for a plurality of gene markers from an organism, and comprising a plurality of alleles for each gene marker; generating a set of machine learning models for each gene marker in the gene marker set configured to predict an allele value for a gene marker when sequence data for that gene marker is missing or unusable; receiving whole-genome sequence data for the organism, comprising missing or unusable sequence data for a gene marker in the plurality of gene markers; analyzing, using the set of machine learning models, the received whole-genome sequence data to determine one or more probable allele values for that gene maker; and displaying the one or more probable allele values.

### FIELD OF THE DISCLOSURE

The present disclosure is directed generally to methods and systems for multi-locus sequence typing using whole genome sequence data.

### BACKGROUND

Hospital-acquired infections result in 100,000 deaths per year, and bacterial infections are becoming increasingly difficult to treat. In 2011, the U.S. National Institutes of Health Clinical Center experienced an outbreak of carbapenem-resistant Klebsiella pneumoniae that affected 18 patients, 11 of whom died. Accordingly, appropriate pathogen surveillance must be applied to prevent the spread of multidrug resistant pathogen within or across healthcare systems.

Effective surveillance relies on the availability of rapid, cost-effective, and informative typing methods to monitor bacterial isolates. While PCR-based typing assays are fast and inexpensive, these tests are incapable of differentiating organisms past the sub-species level due to technological limitations.

Traditionally, multi-locus sequence typing (MLST) has been used for precise molecular identification of bacteria. Specific gene markers (commonly seven genes in total), are sequenced and identified by mapping back to a database with known allele sequences. The combination of these seven gene markers' alleles determines the sequence type (ST) of a particular bacterium. However, this methodology also has its limitations especially when attempting to differentiate genetically related isolates within a single sequence type.

Recently, whole-genome sequencing (WGS) has become increasingly affordable and thus scalable for clinical use. WGS is not limited to seven genes, but rather can be used to examine all polymorphic gene markers, typically spanning 150 to 800 genes for common hospital pathogens. Using high-resolution whole genome MLST (wgMLST) techniques, hospitals can recognize genetic relationships between epidemiologically associated isolates, and can recognize isolates that potentially have the same infection source, as a scalable system for the detection and tracking of the spread of pathogens within a given hospital or healthcare system.

However, wgMLST has limitations as well. Due to the technical and biological complexity, there is a possibility that one or more wgMLST gene markers are missing from the sequencing data of an isolate, or do not have sufficiently high quality sequencing data for an analysis. When this happens, typing may be inaccurate or impossible.

### SUMMARY OF THE DISCLOSURE

There is a continued need for methods and systems that enable accurate multi-locus sequence typing from whole genome sequencing data even when one or more gene markers are missing from the sequencing data or do not have sufficiently high quality sequencing data for typing.

The present disclosure is directed to inventive methods and systems for multi-locus sequence typing using whole genome sequence data. Various embodiments and implementations herein are directed to a method and system that analyzes whole genome sequencing data obtained from a microorganism. The system receives gene marker sets for a plurality of organisms from a database, each gene marker set comprising a plurality of alleles for each gene marker in the set. Machine learning models are created for each gene marker in the gene marker set, and are trained to predict an allele value for a gene marker when sequence data for that associated gene marker is missing or unusable from the whole-genome sequence data. The predicted allele value for the gene marker with missing or unusable sequence data is based at least in part on one or more allele values for one or more of the remaining gene markers in the gene marker set. Once whole genome sequence data is received from an isolate of the organism, and comprises missing or unusable sequence data for a gene marker, the machine learning models for the gene marker with missing or unusable sequence data is used to determine two or more probable allele values for that gene marker. The determined two or more probable allele values for the gene marker are then displayed to a user via a user interface, along with a ranking of those determined two or more probable allele values.

Generally in one aspect, is a method for sequence typing using whole-genome sequence data is provided. The method includes: (i) receiving a plurality of gene marker sets from a database of gene marker sequence data, wherein each gene marker set comprises sequence data for a plurality of gene markers from an organism, the plurality of gene marker sets comprising a plurality of alleles for each gene marker; (ii) generating a set of machine learning models for each gene marker in the gene marker set, wherein each set of machine learning models is configured to predict an allele value for the associated gene marker when sequence data for that associated gene marker is missing or unusable from whole-genome sequence data obtained from the organism, wherein the predicted allele value for the gene marker with missing or unusable sequence data is based at least in part on one or more allele values for one or more of the remaining gene markers in the plurality of gene markers; (iii) storing the generated set of machine learning models for each gene marker in the gene marker set in a database; (iv) receiving whole-genome sequence data for an isolate of the organism, wherein the received whole-genome sequence data comprises missing or unusable sequence data for a gene marker in the plurality of gene markers; (v) analyzing, using the set of machine learning models for the gene marker with missing or unusable sequence data, the received whole-genome sequence data to determine one or more probable allele values for that gene maker; and (vi) displaying, using a user interface, the determined one or more probable allele values for the gene maker with missing or unusable sequence data; where the gene marker set comprises a plurality of predetermined gene markers used for sequence typing one or more organisms.

According to an embodiment, the display comprises a ranking of two or more probable allele values, the ranking based at least in part on a confidence value created by the machine learning models for each of the determined one or more probable allele values. According to an embodiment, the display comprises a confidence value created by the machine learning models for each of the determined one or more probable allele values, a sequence type value for each of the determined one or more probable allele values, and/or an Area Under Curve (AUC) value for each of the determined one or more probable allele values.

According to an embodiment, the method further includes receiving, from a user via a user interface, one or more parameters for one or more of the set of machine learning models.

According to an embodiment, the method further includes generating one or more quality metrics for one or more of the sets of machine learning models. According to an embodiment, the method further includes reviewing, by a user, the generated one or more quality metrics for a set of machine learning models, and adjusting, by the user, one or more parameters of the set of machine learning models.

According to an embodiment, a number of machine learning models in each set of machine learning models corresponds to a number of alleles in the received plurality of alleles for the corresponding gene marker.

According to an embodiment, each set of machine learning models comprises a conserved allele sequence for the corresponding gene marker, and wherein one or more features in each set of machine learning models are calculated based at least in part on SNP differences between an allele and the conserved allele sequence.

According to an aspect is a system for sequence typing using whole-genome sequence data. The system includes: training sequence data comprising a plurality of gene marker sets, wherein each gene marker set comprises sequence data for a plurality of gene markers from an organism, the plurality of gene marker sets comprising a plurality of alleles for each gene marker; whole genome sequence data obtained from an isolate of the organism, comprising missing or unusable sequence data for a gene marker in the plurality of gene markers; a processor configured to: (i) generate a set of machine learning models for each gene marker in the gene marker set, wherein each set of machine learning models is configured to predict an allele value for the associated gene marker when sequence data for that associated gene marker is missing or unusable from whole-genome sequence data obtained from the organism, wherein the predicted allele value for the gene marker with missing or unusable sequence data is based at least in part on one or more allele values for one or more of the remaining gene markers in the plurality of gene markers; and (ii) analyze, using the set of machine learning models for the gene marker with missing or unusable sequence data, the whole-genome sequence data to determine one or more probable allele values for that gene maker; and a user interface configured to display the determined one or more probable allele values for the gene maker with missing or unusable sequence data; where the gene marker set comprises a plurality of predetermined gene markers used for sequence typing one or more organisms.

According to an embodiment, the processor and user interface are further configured to receive, from a user, one or more parameters for one or more of the set of machine learning models.

According to an embodiment, processor is further configured to generate one or more quality metrics for one or more of the sets of machine learning models.

According to an embodiment, the processor and user interface are further configured to receive, from a user, an adjustment of one or more parameters of the set of machine learning models based on the user's review of the generated one or more quality metrics.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for multi-locus sequence typing using whole genome sequence data, in accordance with an embodiment.

FIG. 2 is a schematic representation of a machine learning model analysis and a confidence scoring system, in accordance with an embodiment.

FIG. 3 is a schematic representation of a display of the results of multi-locus sequence typing using whole genome sequence data according to the methods and systems described herein, in accordance with an embodiment.

FIG. 4 is a flowchart of a method for multi-locus sequence typing using whole genome sequence data, in accordance with an embodiment

FIG. 5 is a schematic representation of a system for multi-locus sequence typing using whole genome sequence data, in accordance with an embodiment.

### DETAILED DESCRIPTION OF EMBODIMENTS

The present disclosure describes various embodiments of a system and method for multi-locus sequence typing from whole genome sequencing data even when one or more gene markers are missing from the sequencing data or do not have sufficiently high quality sequencing data for typing. More generally, Applicant has recognized and appreciated that it would be beneficial to provide a system configured to accurately sequence type using whole genome sequencing data. The system creates machine learning models for each gene marker in a gene marker set used for multi-locus sequence typing. The machine learning models are trained using stored gene marker sets for a plurality of organisms, and are configured to predict an allele value for a gene marker when sequence data for that associated gene marker is missing or unusable from whole genome sequence data. The predicted allele value for the gene marker with missing or unusable sequence data is based at least in part on one or more allele values for one or more of the remaining gene markers in the gene marker set. The trained machine learning models are then stored and can be used to analyze whole genome sequence data received from an isolate of an organism, when the data comprises missing or unusable sequence data for a gene marker. Probable allele values for the gene marker, generated by the machine learning models, are then displayed to a user via a user interface, along with a ranking of those determined two or more probable allele values.

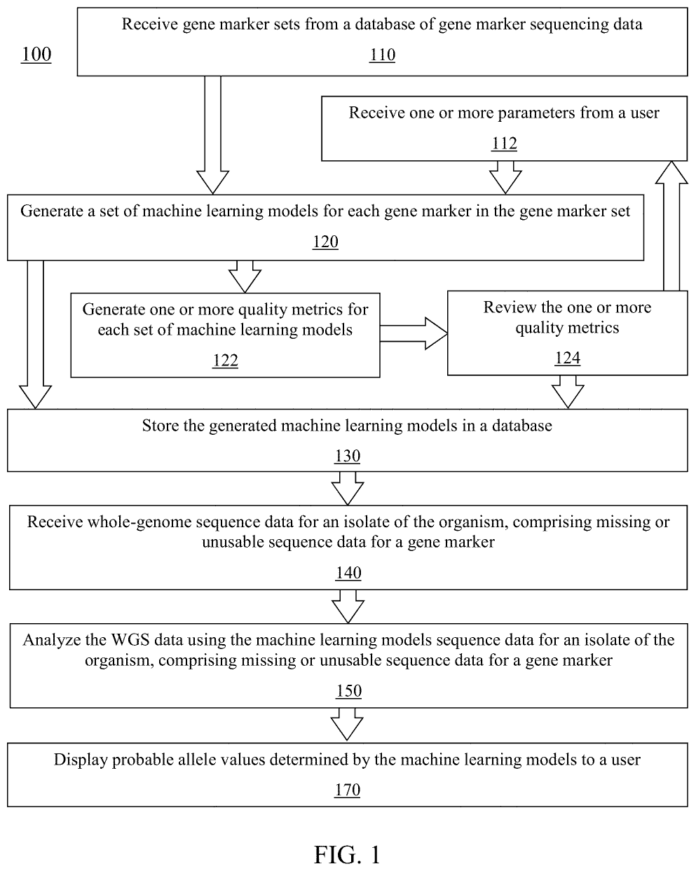

Referring to FIG. 1, in one embodiment, is a flowchart of a method 100 for multi-locus sequence typing from whole genome sequencing data using a multi-locus sequence typing system. The multi-locus sequence typing system may be any of the systems described or otherwise envisioned herein, and may comprise any of the components described or otherwise envisioned herein.

At step 110 of the method, the system receives a plurality of gene marker sets from a database of gene marker sequence data. Each of the gene marker sets includes a plurality of gene markers which are used together to for multi-locus sequence typing, and thus each gene marker set comprises sequence data for the plurality of gene markers from one or more organisms. The sequence data comprises a plurality of alleles for each of the gene marker in the set. Therefore, a gene marker set will comprise the gene markers used for multi-locus sequence typing and the sequence data for those gene markers. For example, a set may comprise the results of multi-locus sequence typing of an isolate of the organism, and thus a plurality of gene marker sets will comprise many different variants for the gene markers.

The database of gene marker sequence data may be any existing or generated database, including local or in-house databases and remote databases. As just one example, the database of gene marker sequence data may be generated by performing multi-locus sequence typing on a large dataset of whole genome sequences. For example, to generate models for multi-locus sequence typing as described herein for an organism such as Klebsiella pneumoniae, whole genome sequencing data for many different variants of Klebsiella pneumoniae can be downloaded from private and/or public sources such as the National Center for Biotechnology Information (NCBI). Multi-locus sequence typing is then performed on each variant, and the results are saved as a gene marker set comprising at least the allele values for each gene marker in the set. The relationships between the gene marker results in a set can then be used to train predictive models.

The gene marker set comprises a plurality of predetermined gene markers used for sequence typing one or more organisms, particularly for whole genome sequence typing of the organism(s). For example, if 325 markers are used for whole genome sequence typing of organism X, a gene marker set will comprise allele data for the 325 markers. The number of markers used for whole genome sequence typing of organism X may be based on community convention, experimentation, or other methods. Accordingly, the system may comprise sequence data for many different gene marker sets for many different organisms.

At optional step 112 of the method, the system receives one or more parameters for generation of the plurality of machine learning models that will be generated by the system. The one or more parameters are received from a user via a user interface. For example, the user can enter one or more expectations of the design and performance of the sets of machine learning models, optionally as a set of constraints such as model size, accuracies, range of coefficients, and/or other constraints.

According to an embodiment, the user can set one or more parameters such that the system will have only a certain number of features in each machine learning model, where the features in a machine learning models can be calculated based on SNP differences between each gene and its corresponding conserved gene. For example, one set of features could be obtained from the SNP difference between Gene 2 (one of the gene markers used for sequence typing) and the conserved sequence of all the corresponding alleles. According to an embodiment, binarized features can be used in the machine learning framework. Thus, for example, if the first quartile, median, and third quartiles of the SNP difference between Gene 2 and the conserved sequence of all the corresponding alleles, SNP_G2, are 10, 20, and 30, the system or user can define the following four features:
Is SNP_G2 less than 10?
Is SNP_G2 greater than or equal to 10?
Is SNP_G2 greater than or equal to 20?
Is SNP_G2 greater than or equal to 30?

Similarly, the system can obtain the other binarized features corresponding to SNP_G1, SNP_G3, and so on.

According to an embodiment, another constraint can be on the range of coefficients. For example, to better understand the interpretable machine learning model, the system or user can set a parameter in which the points (or coefficients) assigned to the binarized features are integers from −5 to 5. Constraints can also be set by the system or user for accuracies including but not limited to Area Under Curve (AUC) and other accuracy or confidence measurements or levels.

At step 120 of the method, the system generates a set of machine learning models for each gene marker in the gene marker set, where each set of machine learning models is configured to predict an allele value for the associated gene marker when sequence data for that associated gene marker is missing or unusable from whole-genome sequence data obtained from the organism. The predicted allele value for that gene marker will be based at least in part on one or more allele values for one or more of the remaining gene markers in the plurality of gene markers. If there are parameters set by the user in step 112 of the method, those parameters are utilized in the generation of the machine learning models. Accordingly, the machine learning framework performs pairwise predictions of each wgMLST allele based on other genes and their alleles.

For example, suppose there are 100 alleles for Gene 1 (one of the wgMLST gene markers used for sequence typing), where an allele represents one of two or more alternative forms of a gene that arose by mutation within the same genetic location (or loci). The first set of machine learning models will be used to predict Gene 1. Next, all the Gene 1 information is removed from the dataset and a set of 100 machine learning models (the same number as that of alleles for Gene 1) is created to predict the allele of Gene 1. Thus, the first machine learning model predicts the probability that Gene 1 was Allele 1, the second machine learning model predicts probability that Gene 1 was Allele 2, and so on.

This way, the system generates approximately 150 to 800 sets of machine learning models (depending on the pathogen and the number of gene markers used for sequence typing that organism) to predict the alleles of the wgMLST gene markers. Fewer or more markers are also possible. For example, the system will generate seven sets of machine learning models to predict the alleles of seven housekeeping genes, and so on.

If the system receives a new bacterial isolate that has one or more gene markers missing, say Gene 2, the Gene 2 set of machine learning models are used, and the allele(s) with the highest calculated probability will be selected for Gene 2.

According to an embodiment, the system generates machine learning models for each of the wgMLST gene markers in which a conserved allele sequence is created from their corresponding alleles in the data set. Features in the machine learning models can then be calculated based on SNP differences between each allele and its corresponding conserved allele.

According to an embodiment, the system uses an interpretable machine learning framework which minimizes logistic loss function with integer coefficients subject to operational constraints such as model size, accuracies, range of coefficients, and/or others. The sets of machine learning models can be risk-calibrated (high reliability) and rank accurate (high AUC) in order to assign confidence (such as a calculated probability for the allele of the missing gene marker) to the sequence typing. The system can, for example, use a mixed-integer programing to solve this NP hard problem, among other approaches. Similarly, the system can obtain the other binarized features corresponding to SNP_G1, SNP_G3, and so on.

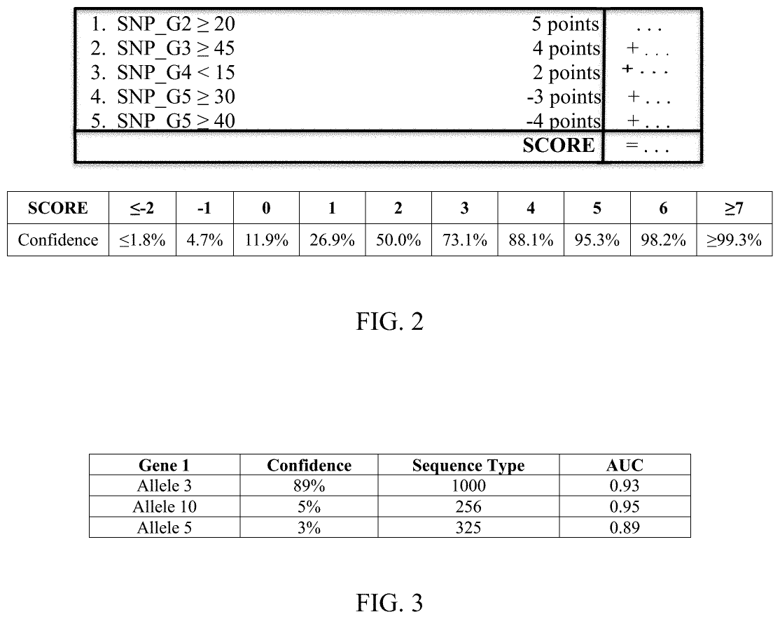

Referring to FIG. 2, in one example, a machine learning model of the set which predicts if Gene 1 is Allele 3, can be determined based on:
SNP_G1 is missing, but:
SNP_G2 is greater than or equal to 20 (therefore the score is granted 5 points);
SNP_G3 is greater than or equal to 45 (therefore the score is granted 4 points);
SNP_G4 is less than 15 (therefore the score is granted 2 points);
SNP_G5 is greater than or equal to 30 (therefore the score loses 3 points);
SNP_G6 is greater than or equal to 40 (therefore the score is loses 5 points);
And so on through the total number of SNPs used in the sequence typing.
   The total final score will depend on the accumulation of points through the entire gene marker set.

According to an embodiment, a confidence score can be generated or calculated using the total final score and a lookup table (such as that shown in FIG. 2) or similar comparison method. For example, if the total final score is 5, the lookup table shown in FIG. 2 will result in a confidence score of 95.3%.

At optional step 122 of the method, the system generates one or more quality metrics for each set of machine learning models. For example, the system can generate quality metric values such as AUC, a reliability diagram, and/or other metrics for the obtained machine learning models. The values or metrics can then be displayed to a user for review.

At optional step 124 of the method, the user receives and reviews the generated one or more quality metrics for each set of machine learning models. If the metric(s) satisfies the reviewing user, the models can be utilized. If the metric(s) fails to satisfy the reviewing user, that user can adjust one or more parameters of a set of machine learning models and the system can proceed back to step 120 to revise or regenerate the adjusted machine learning model(s) and produce new quality metrics for the reviewer to review.

At step 130 of the method, the generated machine learning models are stored in a database such as a machine learning model database. The database can be remote or local, and can be any database or other method of storage. The machine learning models can thus be easily and quickly retrieved when they are needed.

At step 140 of the method, the system receives whole-genome sequence data for an isolate of an organism, in order to generate a sequence typing for the organism. For purposes of this method, the received whole-genome sequence data will comprise missing or unusable sequence data for a gene marker used for sequence typing for the organism and for which a machine learning model exists. For example, the sequence data for the marker may be completely missing or may be corrupted or of insufficient quality to be used for typing. Sequence data that is of insufficient coverage or depth for a gene marker may not be useful or may not be allowed to be used in an analysis. For example, the system may comprise a threshold that sequence data for a gene marker must meet in order to be used for high-quality sequence typing. The sequence data may be generated or received locally, may be downloaded from a remote source, obtained from a database, and/or any other method of generating or receiving whole genome sequence data.

At step 150 of the method, the system analyzes the received whole genome sequence data with the set of machine learning models for the gene marker with missing or unusable sequence data. For example, if sequence data for Gene 1 is missing or unusable, the machine learning models for Gene 1 will be used to analyze the sequence data for the remaining gene markers in the sequence typing gene marker set used for the organism. The system then uses the output of the machine learning models for the gene marker with missing or unusable sequence data to determine one or more probable allele values for that gene marker. According to an embodiment, the system generates multiple possible/probable allele values for that gene marker and ranks two or more of the multiple possible/probable allele values using a ranking system. Thus, the system identifies a determined allele value as the most likely allele value, the second-most likely allele value, and so on. According to an embodiment, the ranking system can be based on a confidence value that accompanies an identified allele value as described or otherwise envisioned herein, among other associated values.

At step 160 of the method the system displays, using a user interface, the one or more probable allele values for the gene marker as determined in step 150 of the method. According to an embodiment, the display can comprise the ranking of two or more determined probable allele values for the gene marker. The number of allele possibilities shown may be determined by the system, such as by comparing the confidence or other score or ranking to a predetermined threshold, or may be determined by a user by setting a confidence or other score or ranking threshold.

According to an embodiment, the display includes a confidence value created by the machine learning models for each of the determined probable allele values, a sequence type value for each of the determined probable allele values, and/or an Area Under Curve (AUC) value for each of the determined probable allele values.

For example, as shown in FIG. 3, there are three allele values determined by the system as being the most likely allele values for the Gene 1 missing or unusable data, namely Allele 3, Allele 10, and Allele 5. Each allele value is associated with a confidence score, a sequence type, and an AUC value (although not all are required). Since Allele 3 has the highest confidence score, for example, it might be ranked the highest. Many other methods of displaying the determined allele values are possible.

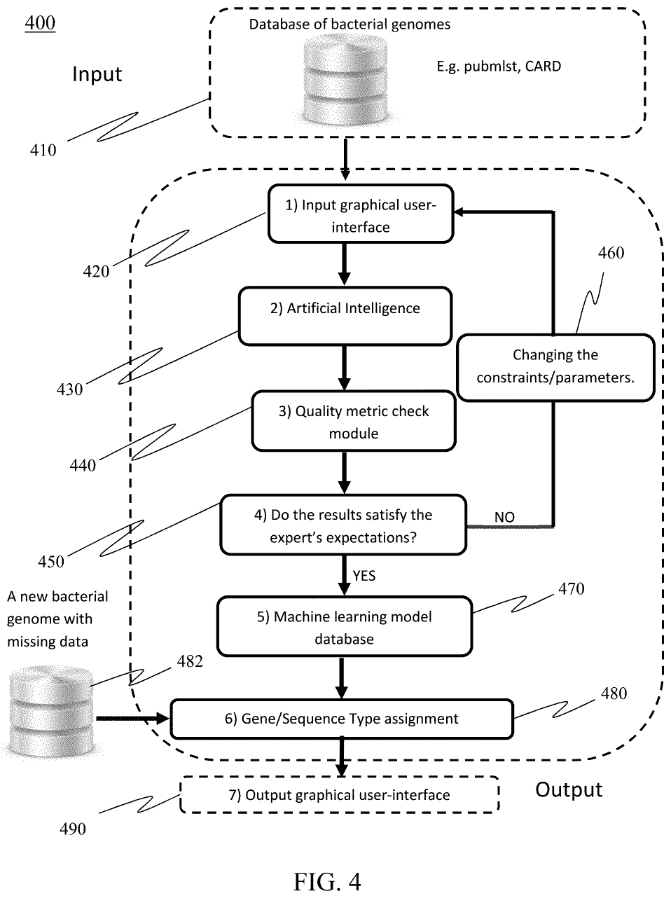

Referring to FIG. 4, in one embodiment, is a flowchart 400 for multi-locus sequence typing from whole genome sequencing data using a multi-locus sequence typing system. The multi-locus sequence typing system may be any of the systems described or otherwise envisioned herein, and may comprise any of the components described or otherwise envisioned herein.

At 410 the system receives, from a database of bacterial genomes, a plurality of bacterial genome sequences with variable allele data. The database may be NCBI, pubmlst, CARD, and any of a plurality of available databases. The database of bacterial genomes may also be generated locally using new or stored sequencing data. The bacterial genome sequences with variable allele data are provided to the multi-locus sequence typing system and used to generate machine learning models.

The system optionally receives input from a user at 420, which may comprise one or more parameters for generation of the plurality of machine learning models that will be generated by the system. The one or more parameters are received from a user via a user interface. For example, the user can enter one or more expectations of the design and performance of the sets of machine learning models, optionally as a set of constraints such as model size, accuracies, range of coefficients, and/or other constraints.

At 430 the system generates a set of machine learning models for each of the gene markers used in sequence typing for one or more organisms, as described or otherwise envisioned herein. The predicted allele value for that gene marker will be based at least in part on one or more allele values for one or more of the remaining gene markers in the plurality of gene markers. If there are parameters set by the user, those parameters are utilized in the generation of the machine learning models. Accordingly, the machine learning framework performs pairwise predictions of each wgMLST allele based on other genes and their alleles.

At 440, a quality metric check module of the system generates one or more quality metrics for each set of machine learning models. For example, the system can generate quality metric values such as AUC, a reliability diagram, and/or other metrics for the obtained machine learning models. The values or metrics can then be displayed to a user for review.

At 450, the user receives and reviews the generated one or more quality metrics for each set of machine learning models. If the metric(s) satisfies the reviewing user, the models can be utilized. If the metric(s) fails to satisfy the reviewing user, that user can adjust one or more parameters of a set of machine learning models at 460 and the system can go back to revise or regenerate the adjusted machine learning model(s) and produce new quality metrics for the reviewer to review.

Once the reviewer approves or fails to revise a machine learning model, the system stores the machine learning model in a machine learning model database at 470. The machine learning model database can be local or remote.

At 480 the system receives from a database 482 whole-genome sequence data for an isolate of an organism, in order to generate a sequence typing for the organism. The received whole-genome sequence data will comprise missing or unusable sequence data for a gene marker used for sequence typing for the organism and for which a machine learning model exists. For example, the sequence data for the marker may be completely missing or may be corrupted or of insufficient quality to be used for typing. Sequence data that is of insufficient coverage or depth for a gene marker may not be useful or may not be allowed to be used in an analysis.

Also at 480, the system analyzes the received whole genome sequence data with the set of machine learning models for the gene marker with missing or unusable sequence data. For example, if sequence data for Gene 1 is missing or unusable, the machine learning models for Gene 1 will be used to analyze the sequence data for the remaining gene markers in the sequence typing gene marker set used for the organism. The system then uses the output of the machine learning models for the gene marker with missing or unusable sequence data to determine one or more probable allele values for that gene marker. According to an embodiment, the system generates multiple possible/probable allele values for that gene marker and ranks two or more of the multiple possible/probable allele values using a ranking system.

At 490, the system displays, using a user interface, the one or more probable allele values for the gene marker. According to an embodiment, the display can comprise the ranking of two or more determined probable allele values for the gene marker. The number of allele possibilities shown may be determined by the system, such as by comparing the confidence or other score or ranking to a predetermined threshold, or may be determined by a user by setting a confidence or other score or ranking threshold.

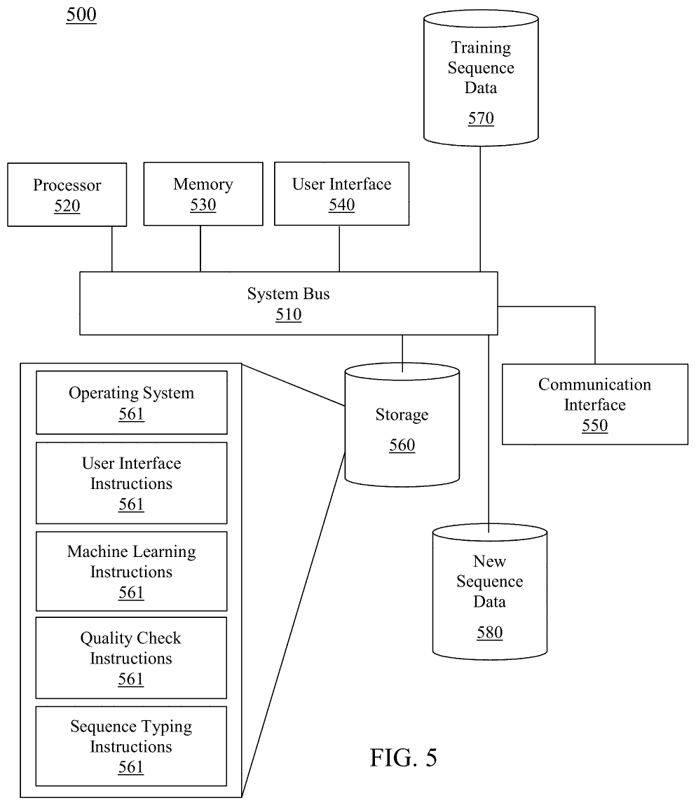

Referring to FIG. 5, in one embodiment, is a schematic representation of a multi-locus sequence typing system 500 for multi-locus sequence typing from whole genome sequencing data when data for an allele is missing. System 500 may be any of the systems described or otherwise envisioned herein, and may comprise any of the components described or otherwise envisioned herein.

According to an embodiment, system 500 comprises one or more of a processor 520, memory 530, user interface 540, communications interface 550, and storage 560, interconnected via one or more system buses 512. It will be understood that FIG. 5 constitutes, in some respects, an abstraction and that the actual organization of the components of the system 500 may be different and more complex than illustrated.

According to an embodiment, system 500 comprises a processor 520 capable of executing instructions stored in memory 530 or storage 560 or otherwise processing data to, for example, perform one or more steps of the method. Processor 520 may be formed of one or multiple modules. Processor 520 may take any suitable form, including but not limited to a microprocessor, microcontroller, multiple microcontrollers, circuitry, field programmable gate array (FPGA), application-specific integrated circuit (ASIC), a single processor, or plural processors.

Memory 530 can take any suitable form, including a non-volatile memory and/or RAM. The memory 530 may include various memories such as, for example L1, L2, or L3 cache or system memory. As such, the memory 530 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 500. It will be apparent that, in embodiments where the processor implements one or more of the functions described herein in hardware, the software described as corresponding to such functionality in other embodiments may be omitted.

User interface 540 may include one or more devices for enabling communication with a user. The user interface can be any device or system that allows information to be conveyed and/or received, and may include a display, a mouse, and/or a keyboard for receiving user commands. In some embodiments, user interface 540 may include a command line interface or graphical user interface that may be presented to a remote terminal via communication interface 550. The user interface may be located with one or more other components of the system, or may located remote from the system and in communication via a wired and/or wireless communications network.

Communication interface 550 may include one or more devices for enabling communication with other hardware devices. For example, communication interface 550 may include a network interface card (NIC) configured to communicate according to the Ethernet protocol. Additionally, communication interface 550 may implement a TCP/IP stack for communication according to the TCP/IP protocols. Various alternative or additional hardware or configurations for communication interface 550 will be apparent.

Storage 560 may include one or more machine-readable storage media such as read-only memory (ROM), random-access memory (RAM), magnetic disk storage media, optical storage media, flash-memory devices, or similar storage media. In various embodiments, storage 560 may store instructions for execution by processor 520 or data upon which processor 520 may operate. For example, storage 560 may store an operating system 561 for controlling various operations of system 500.

It will be apparent that various information described as stored in storage 560 may be additionally or alternatively stored in memory 530. In this respect, memory 530 may also be considered to constitute a storage device and storage 560 may be considered a memory. Various other arrangements will be apparent. Further, memory 530 and storage 560 may both be considered to be non-transitory machine-readable media. As used herein, the term non-transitory will be understood to exclude transitory signals but to include all forms of storage, including both volatile and non-volatile memories.

While multi-locus sequence typing system 500 is shown as including one of each described component, the various components may be duplicated in various embodiments. For example, processor 520 may include multiple microprocessors that are configured to independently execute the methods described herein or are configured to perform steps or subroutines of the methods described herein such that the multiple processors cooperate to achieve the functionality described herein. Further, where one or more components of system 500 is implemented in a cloud computing system, the various hardware components may belong to separate physical systems. For example, processor 520 may include a first processor in a first server and a second processor in a second server. Many other variations and configurations are possible.

According to an embodiment, system 500 comprises or is in communication with a database such as training sequence data database 570 containing a plurality of bacterial genome sequences with variable allele data. The database may be NCBI, pubmlst, CARD, and any of a plurality of available local or remote databases. The database of bacterial genomes may also be generated locally using new or stored sequencing data. The bacterial genome sequences with variable allele data are provided to the multi-locus sequence typing system and used to generate machine learning models.

According to an embodiment, storage 560 of multi-locus sequence typing system 500 may store one or more algorithms and/or instructions to carry out one or more functions or steps of the methods described or otherwise envisioned herein. For example, processor 520 may comprise, among other instructions, user interface instructions 562, machine learning instructions 563, quality check instructions 564, and sequence typing instructions 565.

According to an embodiment, user interface instructions 562 direct the system to receive information from and/or provide information to a user via user interface 540. For example, the user interface instructions 562 may be used to receive one or more parameters for generation of the plurality of machine learning models that will be generated by the system, such as one or more expectations of the design and performance of the sets of machine learning models, optionally as a set of constraints such as model size, accuracies, range of coefficients, and/or other constraints. The user interface instructions 562 also direct the system to provide one or more probable allele values for the gene marker as determined by the system. According to an embodiment, the display can comprise the ranking of two or more determined probable allele values for the gene marker. The number of allele possibilities shown may be determined by the system, such as by comparing the confidence or other score or ranking to a predetermined threshold, or may be determined by a user by setting a confidence or other score or ranking threshold.

According to an embodiment, machine learning instructions 563 direct the system to generate a set of machine learning models for each gene marker in the gene marker set, where each set of machine learning models is configured to predict an allele value for the associated gene marker when sequence data for that associated gene marker is missing or unusable from whole-genome sequence data obtained from the organism. The predicted allele value for that gene marker will be based at least in part on one or more allele values for one or more of the remaining gene markers in the plurality of gene markers. If there are parameters set by the user in, those parameters are utilized in the generation of the machine learning models. Accordingly, the machine learning framework performs pairwise predictions of each wgMLST allele based on other genes and their alleles.

According to an embodiment, quality check instructions 564 direct the system to generate one or more quality metrics for each set of generated machine learning models. For example, the system can generate quality metric values such as AUC, a reliability diagram, and/or other metrics for the obtained machine learning models. The values or metrics can then be displayed to a user for review, such as via the user interface instructions 562. The user interface instructions 562 may also direct the system to receive input from a user regarding the values or metrics, including but not limited changes to one or more parameters for the generation of the machine learning models.

According to an embodiment, sequence typing instructions 565 direct the system to receive new sequence data from a database such as new sequence data database 580. The new sequence data is whole genome sequencing data obtained from an organism for sequence typing. The received whole genome sequencing data is then analyzed with the stored machine learning models to identify one or more probable allele values for a missing/unusable gene marker. According to an embodiment, the system is directed by the sequence typing instructions 565 to generate multiple possible/probable allele values for that gene marker and ranks two or more of the multiple possible/probable allele values using a ranking system.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for sequence typing using whole-genome sequence data, comprising the steps:
receiving a plurality of gene marker sets from a database of gene marker sequence data, wherein each gene marker set comprises sequence data for a plurality of gene markers from an organism, the plurality of gene marker sets comprising a plurality of alleles for each gene marker;
generating a set of machine learning models for each gene marker in the gene marker set, wherein each set of machine learning models is configured to predict an allele value for the associated gene marker when sequence data for that associated gene marker is missing or unusable from whole-genome sequence data obtained from the organism, wherein the predicted allele value for the gene marker with missing or unusable sequence data is based at least in part on one or more allele values for one or more of the remaining gene markers in the plurality of gene markers;
storing the generated set of machine learning models for each gene marker in the gene marker set in a database;
receiving whole-genome sequence data for an isolate of the organism, wherein the received whole-genome sequence data comprises missing or unusable sequence data for a gene marker in the plurality of gene markers;
analyzing, using the set of machine learning models for the gene marker with missing or unusable sequence data, the received whole-genome sequence data to determine one or more probable allele values for that gene maker;
displaying, using a user interface, the determined one or more probable allele values for the gene maker with missing or unusable sequence data;
wherein the gene marker set comprises a plurality of predetermined gene markers used for sequence typing one or more organisms.

2. The method of claim 1, wherein the display comprises a ranking of two or more probable allele values, the ranking based at least in part on a confidence value created by the machine learning models for each of the determined one or more probable allele values.

3. The method of claim 1, wherein the display comprises a confidence value created by the machine learning models for each of the determined one or more probable allele values, a sequence type value for each of the determined one or more probable allele values, and/or an Area Under Curve (AUC) value for each of the determined one or more probable allele values.

4. The method of claim 1, further comprising the step of receiving, from a user via a user interface, one or more parameters for one or more of the set of machine learning models.

5. The method of claim 1, further comprising the step of generating one or more quality metrics for one or more of the sets of machine learning models.

6. The method of claim 1, further comprising the step of reviewing, by a user, the generated one or more quality metrics for a set of machine learning models, and adjusting, by the user, one or more parameters of the set of machine learning models.

7. The method of claim 1, wherein a number of machine learning models in each set of machine learning models corresponds to a number of alleles in the received plurality of alleles for the corresponding gene marker.

8. The method of claim 1, wherein each set of machine learning models comprises a conserved allele sequence for the corresponding gene marker, and wherein one or more features in each set of machine learning models are calculated based at least in part on SNP differences between an allele and the conserved allele sequence.

9. A system for sequence typing using whole-genome sequence data, comprising:
training sequence data comprising a plurality of gene marker sets, wherein each gene marker set comprises sequence data for a plurality of gene markers from an organism, the plurality of gene marker sets comprising a plurality of alleles for each gene marker;
whole genome sequence data obtained from an isolate of the organism, comprising missing or unusable sequence data for a gene marker in the plurality of gene markers;
a processor configured to: (i) generate a set of machine learning models for each gene marker in the gene marker set, wherein each set of machine learning models is configured to predict an allele value for the associated gene marker when sequence data for that associated gene marker is missing or unusable from whole-genome sequence data obtained from the organism, wherein the predicted allele value for the gene marker with missing or unusable sequence data is based at least in part on one or more allele values for one or more of the remaining gene markers in the plurality of gene markers; and (ii) analyze, using the set of machine learning models for the gene marker with missing or unusable sequence data, the whole-genome sequence data to determine one or more probable allele values for that gene maker; and
a user interface configured to display the determined one or more probable allele values for the gene maker with missing or unusable sequence data;
wherein the gene marker set comprises a plurality of predetermined gene markers used for sequence typing one or more organisms.

10. The system of claim 9, wherein the display comprises a ranking of two or more probable allele values, the ranking based at least in part on a confidence value created by the machine learning models for each of the determined one or more probable allele values.

11. The system of claim 9, wherein the display comprises a confidence value created by the machine learning models for each of the determined one or more probable allele values, a sequence type value for each of the determined one or more probable allele values, and/or an Area Under Curve (AUC) value for each of the determined one or more probable allele values.

12. The system of claim 9, wherein the processor and user interface are further configured to receive, from a user, one or more parameters for one or more of the set of machine learning models.

13. The system of claim 9, wherein the processor is further configured to generate one or more quality metrics for one or more of the sets of machine learning models.

14. The system of claim 13, wherein the processor and user interface are further configured to receive, from a user, an adjustment of one or more parameters of the set of machine learning models based on the user's review of the generated one or more quality metrics.

15. The system of claim 9, wherein each set of machine learning models comprises a conserved allele sequence for the corresponding gene marker, and wherein one or more features in each set of machine learning models are calculated based at least in part on SNP differences between an allele and the conserved allele sequence.
